(19) United States
(12) Patent Application Publication
(10) Pub. No.: US 2020/0160996 A1
(43) Pub. Date: May 21, 2020

(54) MULTI-DISCIPLINARY DECISION SUPPORT

(71) Applicant: KONINKLIJKE PHILIPS N.V., EINDHOVEN (NL)

(72) Inventors: Sander Langereis, Mierlo (NL); Jon Ragnar Pluyter, Breda (NL); SeYoung Kim, Eindhoven (NL); David Catharina Petrus Cobben, Manchester (GB)

(21) Appl. No.: 16/615,221

(22) PCT Filed: May 24, 2018
(86) PCT No.: PCT/EP2018/063671

§ 371 Date: Nov. 20, 2019

(30) Foreign Application Priority Data
May 24, 2017 (EP) 17172689.6

(51) Int. Cl. G16H 50/20; G16H 40/60; G16H 50/30; G16H 50/70

(52) U.S. Cl. CPC G16H 50/20; G16H 50/70; G16H 50/30; G16H 40/60

(57) ABSTRACT

The invention relates to clinical decision support tool for supporting a decision based on a plurality of medical findings. The decision to be taken is by a multi-disciplinary team. The medical findings are based on different data sources and integrated to determine a complexity score, based on a consensus and/or conclusiveness of the medical findings. The medical findings and the complexity of the decision are displayed. A circular icon is used to display this information in an intuitive and simple manner. Multiple icons can be combined to arrive at a complexity score of a TNM staging decision.

### FIELD OF THE INVENTION

The present invention relates to the field of decision support, and more specifically but not exclusively, to clinical decision support systems (CDSS).

### BACKGROUND OF THE INVENTION

Modern healthcare decisions on a patient or subject are often fraught with difficulty and complexity. In the modern era with digital equipment, communications and data, the decision-maker can be swamped with vast amounts of data, which can result in an information overload where the most relevant data might be overlooked or missed. Furthermore, advances in medical science and knowledge are constantly expanding and are growing rapidly. This scientific explosion has meant that new specialisms have arisen and where medical specialists or experts focus their learning on a deeper understanding of a specific body of medical knowledge.

In some cases, not only have existing medical specialties deepened and narrowed, but new specialist fields are constantly arising. In turn, each of these specialisms is developing and drawing on new diagnostic modalities.

Another issue that often needs to be taken into account is that the health condition of a subject may be multi-faceted and might not be linked to a single measurable or cause. The subject might have several illnesses or afflictions each with its own set of data, diagnoses and treatments—some of which might come into conflict.

There are software tools available to assist medical professions in their decision-making, but these are organized around a specific medical specialty in a “data silo” approach.

There are also user interface systems such as described in US 2006/0242143A1 published on 26 Oct. 2006 that disclose accessing multiple medical images derived from different types of medical imaging systems including at least one repository. A display processor accesses the repository and initiates generation of data representing a composite display image including multiple image windows individually including different medical images derived from corresponding multiple different types of medical imaging systems for a particular anatomic body part of a particular patient.

### SUMMARY OF THE INVENTION

According to one aspect of the invention there is provided a device for supporting a decision. The device comprising a receiver for receiving a plurality of findings, a complexity engine for determining a complexity score of the decision based on the findings and a display for displaying the findings and the complexity score. In another embodiment, the findings are medical findings.

According to another embodiment, wherein the plurality of findings are based on corresponding data sources.

According to yet another embodiment, there is a provided a device for supporting a TNM staging decision. The device comprising a receiver for receiving a plurality of medical findings relating to a plurality of lesions, a complexity engine for determining a complexity score of a decision on each lesion based on the medical findings for that lesion, a display for displaying a plurality of icons that indicate whether the decisions for each lesion is conclusive based on the corresponding complexity score, and wherein the complexity engine is further configured to determine an overall complexity score for the TNM staging decision based a number of the icons that are inconclusive.

According to yet another embodiment, the device further comprising a mapping unit for mapping the icons to corresponding anatomical locations of the lesions and wherein the display is configured to display the icons on an anatomical grid.

According to yet another embodiment, there is provided a method for supporting a decision, the method comprising receiving a plurality of findings, determining a complexity score of the decision based on the findings and displaying the findings and the complexity score.

According to yet another embodiment, there is a provided a computer program for enabling a processor to perform the method.

According to yet another embodiment, there is a provided a device for supporting a decision based on a plurality of medical findings, the device comprising: a receiver for receiving a the plurality of medical findings, each medical finding is received from a different data source; a complexity engine for determining a complexity score indicating a level of certainty of for the decision based on the medical findings from the different data sources; and a display for displaying the medical findings and the complexity score.

These and other aspects of the invention will be apparent from and elucidated with reference to the embodiments described hereinafter.

### BRIEF DESCRIPTION OF THE DRAWINGS

The invention is described by way of example with reference to the following drawings, in which:

FIG. 1 shows a device in a first system;

FIG. 2 shows a device in a second system;

FIG. 3 shows an embodiment of a device for supporting decisions; and

FIG. 4a shows an embodiment of a circular icon;

FIG. 4b shows a further embodiment of a circular icon with medical findings;

FIG. 5 shows a rule engine for determining the complexity of a decision according to one embodiment;

FIG. 6 shows an embodiment of mapping of circular icons onto an anatomical grid;

FIG. 7 shows a lung management cancer management system using TNM staging according to an embodiment of the invention;

FIG. 8 shows a block diagram of a system according to an embodiment;

FIG. 9 shows is a simplified block diagram of a system according to an embodiment; and

FIG. 10 is a simplified block diagram of a computer within which one or more parts of an embodiment may be employed.

### DETAILED DESCRIPTION OF THE EMBODIMENTS

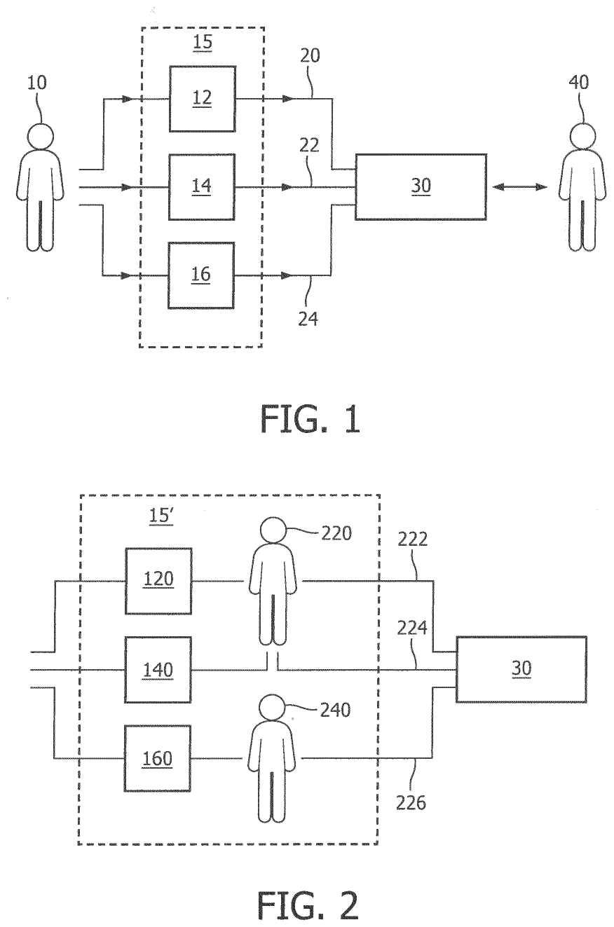

The device 30 and the different systems in which the device may be used, as shown in FIGS. 1 and 2 respectively, are examples only.

The device is a clinical decision support unit in that it supports the decision of a medical practitioner 40 or a multi-disciplinary team of healthcare professionals. Modern healthcare decisions are often made by a multi-disciplinary team. In particular, the device outputs a complexity indication or score that takes into account the medical findings from multiple different diagnostic modes (data sources). A simple example is a lung tumour that might be detectable by pathology, but not by x-ray. The medical findings might also differ where a specialist needs to interpret the results from a particular data source or where data from that data source is insufficient.

A complexity score according to some embodiments can be used to take into account some or all of the following considerations:
each disease might have a different set of medical findings associated with it,
some medical findings might be more relevant to that particular disease than others,
some medical findings might have different levels of certainty (conclusiveness) than others,
some medical findings might contradict each other, i.e. the level of agreement (consensus) between the medical findings might differ.

Faced with such overwhelming information from multiple data sources and medical findings, a support device according to an embodiment of this invention is able to integrate all this information and display a quick and objective assessment of the complexity score of each medical case.

There are several other advantages associated with providing such a complexity score. For example, the clinician can save time in using the complexity score to quickly identify the amount of time and care they need to spend on a medical case in reaching a diagnosis. The higher the complexity score, the more closely a medical case for a particularly subject needs to be scrutinized and the more time spent on it. Another example is tumour board meeting, which might consist of a group of clinicians from different specialities that meet to discuss the medical cases associated with a plurality of subjects. The complexity score from the support tool allows for the medical cases to be ranked according to their complexity (eg from most complex to lease complex), which ensures that the most complex cases are discussed first where concentration spans are at the highest or where the time allocated to the meeting might require priority for the more complex cases (i.e. triage). It also introduces objectivity into the decision support, in that whereas a clinician from one specialty might diagnose a disease as benign, a clinician from a different specialty might diagnose the same disease as malignant. The complexity score means the clinicians have to step back from their own discipline and consider other disciplines. If nothing else, a support device according to some embodiments can be used to the clinician to sense-check their own decision or diagnosis. More specifically, the support device automatically takes into account a wider range of data sources that might have not been considered by a particular clinician. It also means that less experienced physicians, who might subjectively differ on interpretation of the results from different data sources, might benefit from a support device with a complexity score whose weightings can be programmed according to “best practices” to offer more objective guidance. For example, the complexity indicator could be programmed or setup a-priori by a large team of experts from a different specialties to arrive at a optimally configured complexity indication for a particular disease and the medical findings associated with that disease. Also, in some embodiments the complexity score can be presented in a way that allows the clinician to quickly understand which medical findings or data sources are more relevant to the complexity score, which allows the clinician a better understanding of the relevant weighting given to each medical finding in arriving at the complexity indication.

A further advantage is to better support the diagnosis of a disease such as cancer, which might affect multiple different body parts, and where each body part might have a different complexity score associated with it based on multiple medical findings for that body part. The support device according to some embodiments is able to determine an overall complexity score that takes into account the magnitude of the different complexity scores associated with the different body parts. All this information is integrated and reflected in an overall complexity indication, which allows a clinician to instantly and intuitively understand the complexity of the decision based on all the information available from multiple medical findings associated with multiple body parts.

The system in FIG. 1 is shown to comprise a subject 10 that has a medical condition, which needs to be diagnosed and/or treated by a medical practitioner 40. In the example of FIG. 1, the subject 10 has a possible lung tumor and medical staff wish to confirm if it is cancerous.

There are a number of different diagnostic modes (i.e. data sources) that can be used to arrive at a diagnosis. For example, one mode of diagnosis might be histology where a physical biopsy or tissue sample of the lung tumor is taken. The tissue sample is then studied under a microscope or similar equipment for diagnosing the disease in question.

A pathologist is a medical specialist that assesses and interprets the data coming from the histology tests. An endoscopic scan might involve a medical specialist simply giving a lesion a visual scan for irregularities without taking a biopsy. A medical specialist might also take into account the symptoms experienced by the subject, albeit with less of a weighting as compared to the clinical data from the biopsy or samples. The medical specialist might also be for example, an oncologist, pulmonologist, surgeon, etc. depending on tumor type, country, hospital preferences, etc.

Insofar as imaging is concerned, there are also a plurality of imaging diagnostic modes. These might include x-ray, which uses x-rays to get an image of the structure of the internal organs and tissues of the body. A computerized tomography (CT) scan is a series of slices of x-rays analyzed and rendered by a computer to provide a visual display of a lesion from a number of different perspectives. A magnetic resonance imaging (MRI) scan uses magnetic waves rather than x-rays to image a lesion. Ultrasound uses sound or acoustic waves to construct an image of a lesion.

A positron emission tomography (PET) scan fires positively charged particles at a lesion to determine its chemical activity. Unlike MRI, CT and X-ray that are concerned with imaging the structure of a lesion, the PET scan is concerned with imaging the function (chemical and metabolic) of a lesion.

A radiologist is the medical specialist that assesses and interprets the data coming from the various imaging data sources, such as X-ray, PET, CT, ultrasound, etc.

There are also other diagnostic modes where there is an overlap in specialties or where other specialists are needed to interpret the data from other diagnostic mode being used. For example, endoscopic ultrasound (EUS) is a diagnostic mode that combines endoscopy with ultrasound, which allows for visualization of the structure and performing a minimally-invasive biopsy. It requires the skills of a radiologist and a pathologist. Consequently, there is a specialty for assessing and interpreting the data from EUS scans.

FIG. 1 shows a number of data sources 12, 14 and 16 from which medical findings 20, 22 and 24 are produced. The data sources 12, 14 and 16 represent the different diagnostic modes or data sources used to diagnose a patient. FIG. 2 shows in more detail an example of another system where the data from the data sources 120, 140, 160 is interpreted by a medical specialists 220 and 240. For example, data sources 120 and 140 could represent data from x-rays and PET scans respectively. In the embodiment of FIG. 2, the medical specialist 220 is a radiologist. The data source 160 is data from histology or cytology tests, which is interpreted by a different medical specialist 240, who is a pathologist. It will be appreciated that different combinations are possible and that these are examples only. Specialisms can be combined or further differentiated. It will also be appreciated that while FIG. 2 is a system where a medical specialist is needed, in yet a further embodiment the interpretative or diagnostic function might be performed by a machine, which is integrated into the data source.

The device 30 receive a plurality of medical findings, shown by lines 20, 22 and 24 in FIG. 1 and by lines 222, 224 and 226 in FIG. 2. These medical findings might be diagnoses from different specialists based on different data sources. For example, diagnosis 20 resulting from a histology data source 12 is that the lung tumor is malignant (cancerous). However, the diagnosis 22 resulting from an EUS scan data source 14 is that the lung tumor is benign (non-cancerous). Finally, the diagnosis 24 resulting from a PET scan data source 16 is that it is inconclusive from the PET diagnostic mode whether the tumor is malignant or benign. Faced with these different medical findings, the medical practitioner or team 40 needs to try reach consensus on the decision as to the overall diagnosis for the patient.

In one embodiment of the invention, the device 30 is able to receive the medical findings from a plurality of data sources or diagnostic modes. Instead of the device 30 only providing support to single specialty, or based on a single data source, an embodiment of the invention is able to draw on multiple specialties and/or data sources to arrive at a more informed decision as to supporting the diagnosis or treatment of the subject. The advantage of this embodiment is that the medical practitioner 40 is able to draw on a wider range of data to support its decision.

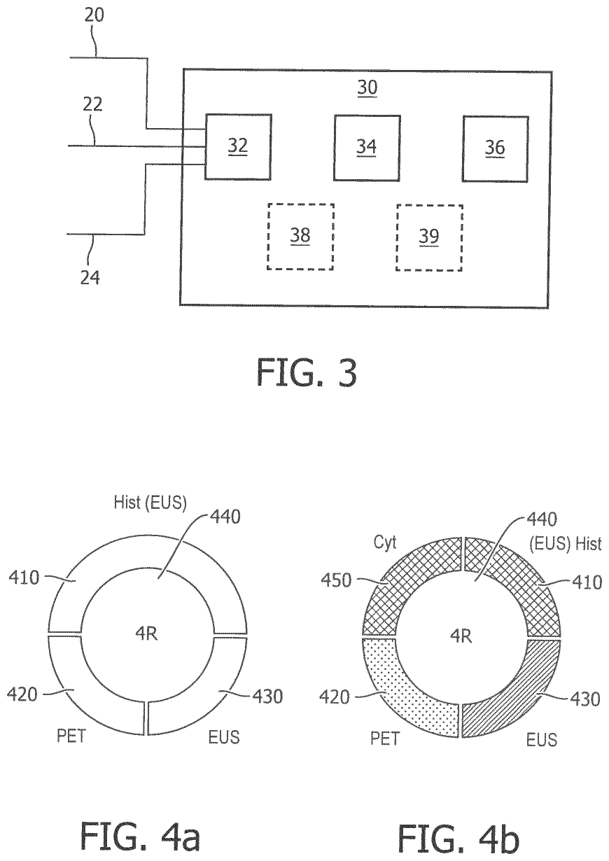

FIG. 3 shows an embodiment of the device 30, which comprises a receiving unit 32, a complexity engine 34 and a user interface 36. It also shows that a memory unit 38 and a mapping unit 39 are optionally included. In another embodiment, the memory unit 38 and the mapping unit 39 are not necessary.

The memory unit 38 could be a database for storing medical findings and/or a computer program for implementing the functionality of device 30. More generally, it will also be appreciated by the skilled person that memory, processing and/or any other computing resources, which might be used to implement the various units of the device, could be located remotely from the device 30. For example, cloud-computing could be used to access computing resources remotely over a network. In yet another embodiment, the functions performed by the receiving unit 32, complexity engine 34 and user interface 36 are all combined into one unit.

In one embodiment, the receiving unit 30 is configured to receive the plurality of medical findings 20, 22 and 24 over a wireless link. Alternatively, such medical findings could be communicated over fixed media, such as via cable or fiber optics. In another embodiment the medical findings 20, 22 and 24 are received over the IT network of a hospital, but such medical findings could also be conveyed from more remote locations using the Internet.

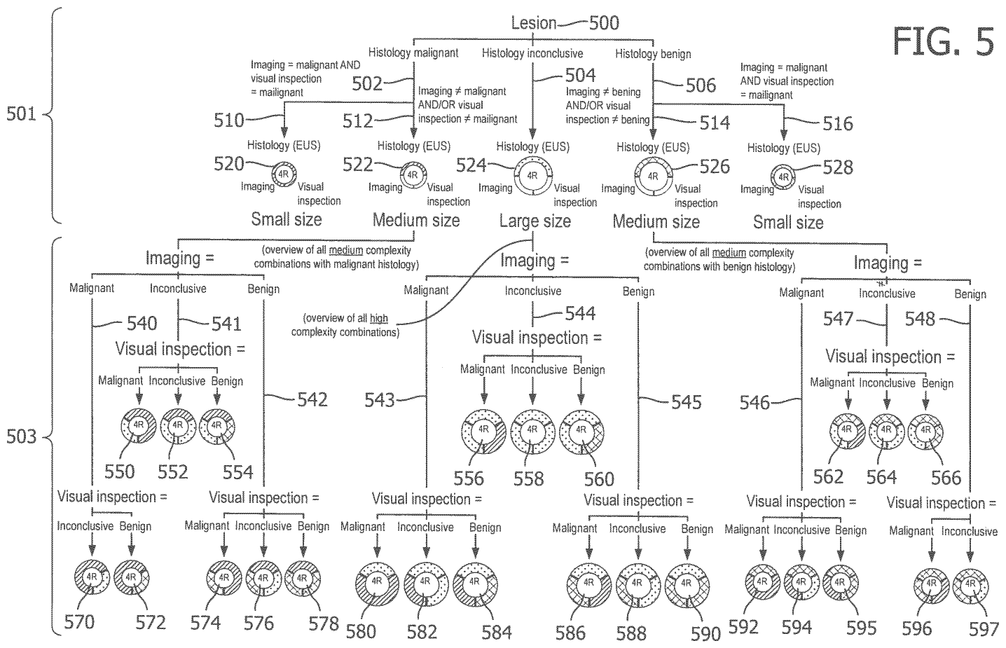

In one embodiment, the complexity engine 34 is for determining a complexity score of the decision based on conclusiveness and/or consensus of the medical findings. The user interface 36 has a display for displaying the medical findings and the complexity of the decision. In one embodiment, the displaying of the medical findings and complexity score of the decision is indicated by a circular icon as shown in FIGS. 4a and 4b.

Specifically, FIGS. 4a and 4b show a circular icon with medical findings that correspond to three, and four, different data sources respectively. Specifically, FIG. 4a shows lymph node 4R of the subject, who has been investigated using histology 410, PET 420 and EUS 430 data sources. FIG. 4b shows the same lymph node 4R, but which has additionally been examined using a cytology 450 data source. FIG. 4b also shows that the medical findings, which correspond to each of the data sources, are displayed using different fill patterns in corresponding segments of the circular icon. For example, a horizontal fill pattern in segments 410 and 450 indicates that the medical findings based on cytology and histology are malignant. The vertical fill pattern in segment 430 indicates that the medical findings based on EUS are benign. The dotted fill pattern in segment 420 indicates that the medical findings based on PET are inconclusive.

Although FIG. 4b shows an embodiment where the segments are differentiated by fill patterns, it will be appreciated this might alternatively be done using gray-scale settings. Such embodiments might be advantageous for black & white or monochrome displays. In yet another embodiment, the segments of the circular icon are color-coded with a red color indicating malignant, a green color indicating benign and a yellow color indicating an inconclusive finding. This embodiment advantageously allows a user to intuitively and simply appreciate what the medical findings are that correspond to each data source, without having to continuously refer to a legend or key to understand their meaning.

FIG. 5 shows a rule engine for determining the size of a circle (or the complexity of a decision) according to one embodiment of the invention. It will be appreciated that other embodiments could implement the functionality of this rule engine using other methods, such as using a look-up table or a weighting function, which are applied to the medical findings.

FIG. 5 is an example where a lesion 500 has been investigated using three different diagnostic modes (i.e. three different data sources): histology, imaging and visual inspection. Imaging in this embodiment might mean radiological imaging such as an x-ray, whereas visual inspection might be where a specialist has assessed the lesion with the naked eye for irregularities.

The rule engine of the embodiment in FIG. 5 assumes that histology is the most accurate diagnostic mode. In other words, more weight is given to the medical finding relating to histology, as compared to the medical findings based on the other data sources (imaging and visual inspection).

The top-half 501 of FIG. 5 shows circle icons 520, 522, 524, 526 and 528 with different sizes. The size of each circle indicates a level of complexity or complexity score of the decision.

For example, a small-sized circle indicates the decision has low complexity, which arises where:

a) the medical findings are conclusive; and
b) there is consensus of the medical findings, which means that the probability of a false positive is low.

Small-sized circles 520 and 528 show the two circumstances when this might arise, which in the case of circle 520 is when all the medical findings indicate that node 4R is malignant, whereas in the case of circle 528 is when all the medical findings indicate that node 4R is benign. A small icon, as displayed on the user interface 36, indicates to a user 40 that this diagnostic decision is reasonably certain. Therefore, not a lot of time needs to be spent by, for example, a multi-disciplinary team analyzing this decision.

A medium-sized circle indicates the decision has medium complexity, which arises where the pathology medical findings are conclusive, but:

a) the medical findings of at least one of the other data sources (imaging or visual inspection) are inconclusive; and/or
b) there is not consensus of the medical findings (i.e. at least one of the medical findings is different). Therefore, the probability of a false positive and a misdiagnosis is medium. For example, if histology is benign (negative) then there is a risk of a false negative, whereas if histology is malignant (positive) then there is a risk of a false positive.

Medium-sized circles 522 and 526 show, with respective lines 540, 541, 542 and lines 546, 547, 548 to the bottom-half 503 of FIG. 5, the many different combinations where a medium complexity decision arises. Specifically, circle 522 is where the medical finding based on histology is malignant and the bottom half 503 of FIG. 5 shows medium circles 550, 552, 554, 570, 572, 574, 576 and 578 with different combinations of the medical findings based on imaging and visual inspection data. Circle 526 is where the histology based on histology is benign and the bottom half 503 of FIG. 5 shows medium circles 562, 564, 566, 592, 594, 595, 596 and 597 with different combinations of the medical findings based on the imaging and visual inspection. These various medium complexity decisions arise when the medical findings based on imaging or visual inspection are inconclusive and/or there is not consensus. A medium icon displayed on the user interface is used to indicate that this decision requires more time for discussion by the multi-disciplinary team, as compared with a small icon, but not as much as compared with a large icon.

A large-sized circle indicates that the decision has high complexity, which arises whenever the medical findings relating to the pathology are inconclusive. This demonstrates the high weighting that is given to pathology as a diagnostic mode for cancer. If the medical finding from pathology is inconclusive, the decision will be high complexity, irrespective of whether or not the medical findings from the other data sources are all conclusive and in consensus. The bottom half of FIG. 5 shows various large icons 556, 558, 560, 580, 582, 584, 586, 588 and 590 with different combinations of the medical findings from the imaging and visual inspection data sources. A large icon is used to indicate that these are the most uncertain decisions, which require the most time and discussion from the multi-disciplinary team 40.

It will be appreciated that in a different embodiment, a number can be used to reflect the complexity score of a decision, rather than sizing the circle. The advantage of the embodiment in which the circle is sized, is that it avoids the need for text or numbers or legends, which a user might need to look up. Therefore, the sizing of the icon advantageously contributes to the intuitive and simple manner in which this information is conveyed. In other words, the user is not only being provided with an integrated set of medical findings from a plurality of data sources in a graphical manner, but also the size indicates the complexity score of the decision in a graphical manner that avoids cluttering of the display or overloading the multi-disciplinary team 40 with information.

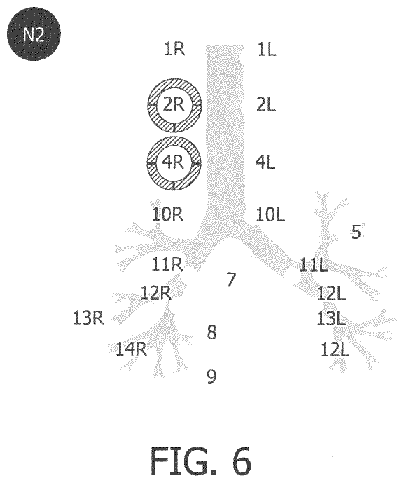

FIG. 6 shows the mapping of circular icons onto an anatomical grid. In this embodiment, the mapping unit 39 is needed for locating the circular icons to lesions of the subject's anatomy.

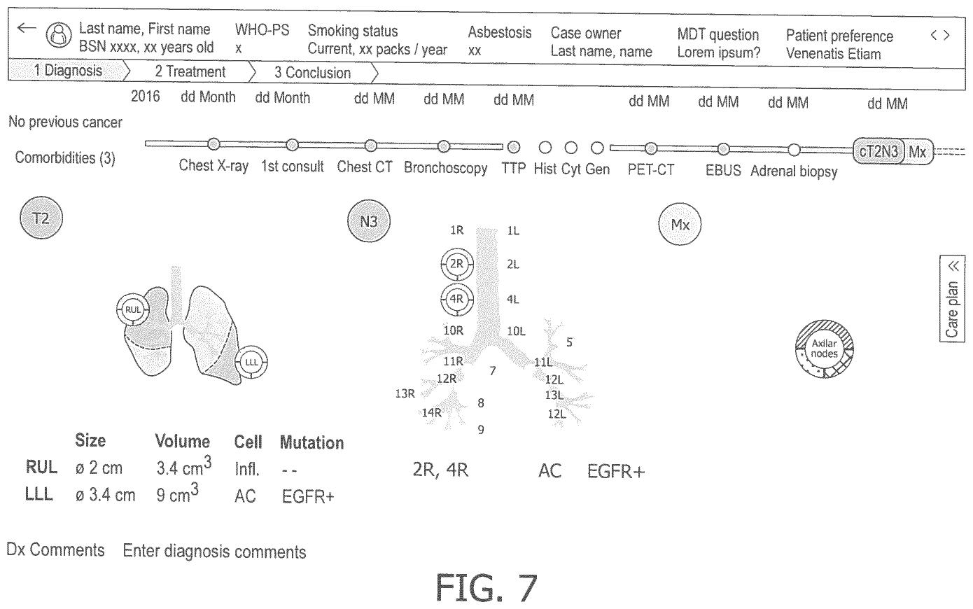

This circular icon embodiment might also be advantageous where it displays a plurality of different sized icons corresponding to different lesions, each loaded with their own dense yet unique information, which is intuitively and efficiently conveyed to a user 40 of the user interface. If the user is multi-disciplinary team, the team can quickly triage which decisions (large circles) they need to spend the most time discussing and why. TNM staging describes the extent and level of advancement of a subject's cancer. By identifying the correct TNM staging, the relevant treatment plan can be timed to start as appropriate, such as surgery, chemotherapy and/or radiotherapy. In yet another embodiment, the mapping to the anatomical grid in FIG. 6 helps the multi-disciplinary team 40 to derive a TNM stage diagnosis decision based on a set of circular icons representing all data sources relevant to the subject. For example, the involvement of malignant mediastinal lymph nodes (e.g., 2R, 4R) indicates an N2 classification as shown in FIG. 6. The anatomical mapping of the mediastinum is used for determining the correct N-stage classification. FIG. 7 shows how this is done for T2 (tumor), N3 (node), and Mx (metastases) to help with the TNM staging diagnosis. The current practice is that medical specialists have to make this anatomical mapping in their mind, and hence diagnosing a TNM stage is error prone. The mapping of this embodiment to support the medical specialist in diagnosing the correct TNM staging overcomes these disadvantages.

In another embodiment, the set of circular icons is used to calculate the relative complexity of TNM staging decision. Relative complexity of TNM staging is a function of the number of circular icons and the relative number of inconclusive circular icons. The complexity C of a TNM staging decision according to one embodiment is calculated using the equation:

C=x*(I+((y+2z)/x))

where:

x=the number of circular icons that are not conclusively benign or malignant (i.e. total number of icons—low complexity icons)

y=the number of medium complexity circular icons (i.e indicates a lack of consensus); and

z=the number of high complexity circular icons (i.e. indicates a lack of consensus with possibly lack of reliable information to make a decision)

Some example scenarios of complexity scores are presented in Table 1 below:

TABLE 1

| Complexity | x | y | z |
|---|---|---|---|
| 16 | 7 | 5 | 2 |
| 14 | 7 | 7 | 0 |
| 7 | 7 | 0 | 0 |
| 12 | 5 | 3 | 2 |
| 10 | 5 | 5 | 0 |
| 5 | 5 | 0 | 0 |
| 4 | 1 | 1 | 1 |
| 2 | 1 | 1 | 0 |
| 1 | 1 | 0 | 0 |

An objective measure of relative complexity of TNM staging decision can be utilized a-priory (i.e., before the staging decision is made). This embodiment would therefore advantageously allow the prioritization of cases to be discussed in a tumor board meeting according to the expected complexity of the decision. For example, to start the tumor board meeting with the estimated most complex cases followed by more straightforward cases, or the other way around.

In a different embodiment, parameter x can be utilized during the discussion of the multi-disciplinary team to give a warning when there are inconclusive lesions remaining (i.e., x>0) that need to be addressed before selecting a treatment. For example, in FIG. 7 there is still inconclusive information about the adrenal nodes.

While embodiments have been described in an oncology setting, it should be appreciated that this ability to provide a-priory estimates of the complexity of multivariate decisions, applies more broadly to any multi-disciplinary context. Therefore, different embodiments of the invention might deal with other cancer types (e.g., colon, breast, prostate), or other stages in the cancer care path, or other diseases, or even fields other than medicine that require the multi-disciplinary coordination of complex cases, such as military and aviation.

In a further embodiment, there is not only support for diagnostic decisions, but also treatment decisions by integrating other healthcare professions that might be involved in that treatment, for example psychologists, nutritionists, physiotherapists, etc.

The current practice of organizing and assessing data in the “data silo” approach of a single medical specialty, is overcome by an embodiment of the invention that advantageously allows integrating data sources from multiple disciplines for improved decision-making about patient care. This embodiment allows integration of data from different medical domains (e.g. radiology, pathology, oncology, radiotherapy) to guide consensus towards diagnosis and treatment selection in a multi-disciplinary setting. An embodiment of the invention provides a visually-based user interface for enabling a multi-disciplinary team to make improved decisions by integrating data silos into actionable clinical information, resulting in an increased standard of care.

Another embodiment of the invention integrates data silos and aggregates related and relevant data, according to a logic that is represented in a visual manner, to guide consensus towards TNM stages in a multi-disciplinary setting, which is expected to improve the quality of the diagnosis and treatment selection made by multi-disciplinary teams.

In an embodiment, the functionality of the device 30 is implemented in software, but in other embodiments, the device 30 might be implemented in hardware using, for example, spatially distributed labeled backlit buttons, cards or paper documents.

An embodiment has an icon with a circular shape, but it should be appreciated that other shaped icons (e.g. square, rectangular, triangular, etc.) could also be used. In yet a further embodiment, the upper half of the circular icon represents pathology findings (cytology, histology, etc.), whereas the lower half of the circular icon represents radiological findings (CT, PET, etc.).

According to an embodiment, the device 30 is not for diagnosing or treating the subject, rather to solve a technical problem of how to support medical decisions that need to integrate different medical findings based on different data sources or specialties.

In an embodiment, the plurality of medical findings relating to a decision, based on corresponding data sources selected from a group that includes at least two of following: histology, cytology, visual inspection, x-ray, endoscopic ultrasound, PET, CT, and MRI, ultrasound.

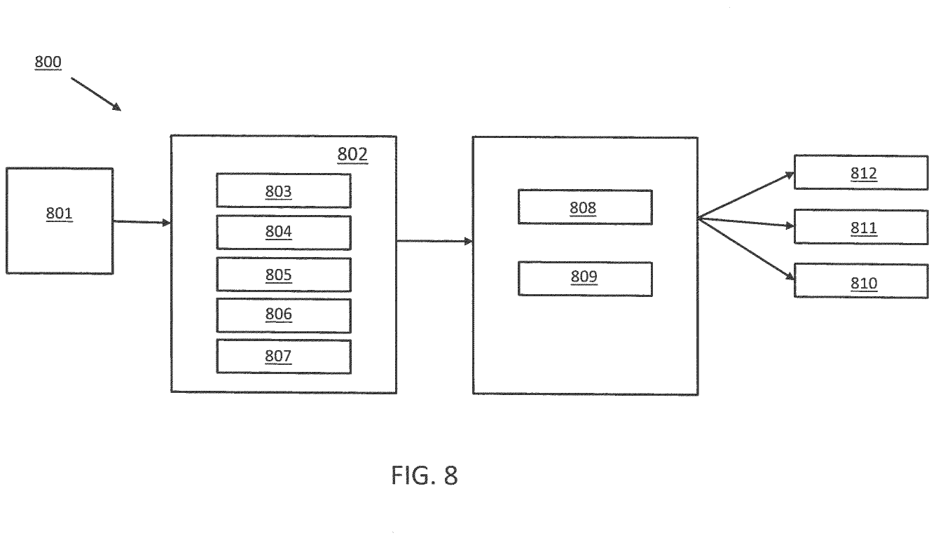

FIG. 8 illustrates a system 800 according to a further embodiment that is able to provide an a-priory assessment of complexity for a medical decision. The system has an input from a database 801, which may include example patient data, diagnostic studies, images, reports, etc. Patient data in database 801 may include demographic information of a patient, for example gender, age, ethnicity, etc. The clinical information in the database 801 may include family history, medical history, symptoms, patient preference, risk factors, etc. The diagnostic studies in the database 801 may relate to one of more patients, including, e.g., CT, PET, MRI, X-Ray, pathology, endoscopy, lab tests, functional tests, etc.

Database 801 provides an input to a complexity engine 802, which in one embodiment comprises a processor 803 for executing computer-executable instructions stored in memory 804. The memory 804 may be a computer-readable medium on which a control program is stored, such as a disk, hard drive, or the like. Common forms of computer-readable media include, for example, flexible disks, hard disks or other tangible medium from which the processor 803 can read and execute. It will be understood that the processor functionality 803 may be implemented on or as one or more general purpose computers, special purpose computer(s), a programmed microprocessor or microcontroller and peripheral integrated circuit elements etc.

In this embodiment, processing block 805 determines the probability of malignancy for each medical finding or data source. For example, the clinical parameters for a particular medical case are identified by processing block 806 which takes into account parameters such as lesion size, suspiciousness for malignancy, lesion location, etc. The processing block 807 represents certain clinical pathway parameters, which might represent the performed versus expected events for a patient in a given clinical pathway, e.g., the performed diagnostic tests versus expected diagnostic tests.

The complexity score 808 is derived from the complexity engine 802 and is based on a programmable algorithm, weightings or table (for example Table 1 as previously described). The embodiment of FIG. 8 also shows a data completeness classifier 809, which indicates a level of the completeness of data from the data sources. This output of the completeness classifier might be in numerical form (eg, a number between 0-100) or categorical (eg, “sufficiently complete for tumor board discussion” and “insufficiently complete for tumor board discussion”). In other words, the clinician is not only provided with a complexity score, but also a data completeness indication, both of which can be provided in numerical, categorical or visual form in different embodiments as would be appreciated by the skilled person.

The complexity engine 802 is able to output the complexity score 808 and data completeness classifier 809 in different ways.

In one embodiment, missing diagnostic information list 810 is generated from the data completeness classifier 609. For example, in a tumor board setting only the cohort of patients with a sufficiently high completeness classifier are prone to be discussed.

In another embodiment, a prioritized lesion list 811 is generated from the complexity score 808, highlighting those lesions with highest complexity that need most attention in the tumor board discussion. This helps the tumour board focus and make optimal use of their time together.

In another embodiment, a ranked patient list 812 is derived using both the complexity score 808 and data complete classifier 809. At a tumor board meeting, the cohort of patient cases to be discussed can be ordered from high to low case complexity to match the declining attention span of the multi-disciplinary team of specialists discussing the case. In another embodiment, when the amount of patients exceeds the time available to discuss all of the patient cases, the system 800 can present to the user the set of patients with sufficient data completeness score and low complexity score, which may not have to be discussed at all in the tumor board but can instead be handled outside a tumor board setting.

The system 800 is an integrated multi-disciplinary support tool, which can be programmed to support various diagnostic and/or treatment tasks. For example, though automated characterization of the nature of abnormalities and/or malignancy based on integrated information), patient stratification and treatment selection (e.g., selecting additional diagnostic tests based on probability of malignancy etc.), or an evaluation task (e.g., assessment of disease progression and/or treatment efficacy).

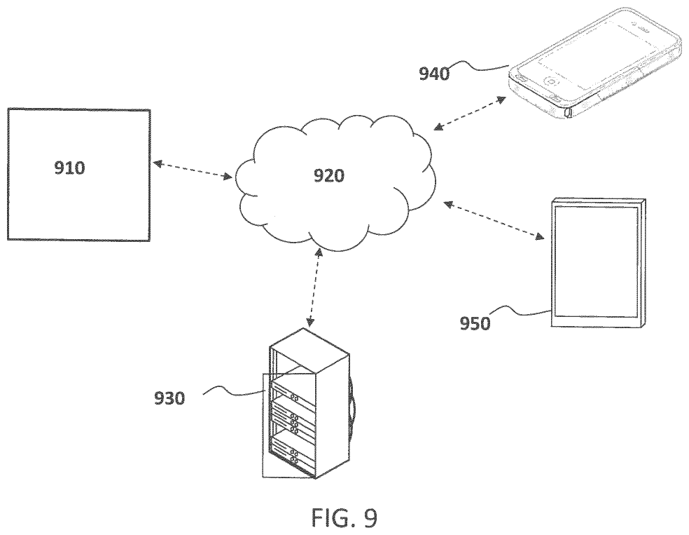

Referring now to FIG. 9, there is depicted an embodiment of a system according to an embodiment of the invention comprising an input system 910 arranged to obtain interaction data associated with a subject.

Here, the input system 910 is adapted to obtain interaction data associated with a subject using a pointing device, the obtained interaction data being representative of the subject's interaction with the pointing device. The input system 910 is adapted to output one or more signals which are representative of obtained interaction data.

The input system 910 communicates the output signals via a network 920 (using a wired or wireless connection for example) to a remotely-located data processing system 930 (such as server).

The data processing system 930 is adapted to receive the one or more output signals from the input system 910 and process the received signal(s) to determine a complexity score associated with the subject. Thus, the data processing 930 provides a centrally accessible processing resource that can receive information from the input system 910 and run one or more algorithms to transform the received information into a complexity score that is representative of the relevance, conclusiveness and/or medical findings of the subject. Information relating to the complexity score can be stored by the data processing system (for example, in a database) and provided to other components of the system. Such provision of information about a subject's complexity score may be undertaken in response to a receiving a request (via the network 920 for example) and/or may be undertaken without request (i.e. ‘pushed’).

For the purpose of receiving information about a subject's complexity score from the data processing system 930, and thus to enable subject-specific information to be viewed, the system further comprises first 940 and second 950 mobile computing devices.

Here, the first mobile computing device 940 is a mobile telephone device (such as a smartphone) with a display for displaying information in accordance with embodiments of the proposed concepts. The second mobile computing device 950 is a mobile computer such as a Laptop or Tablet computer with a display for displaying information in accordance with embodiments of the proposed concepts.

The data processing system 930 is adapted to communicate the complexity score and or data completeness score to the first 940 and second 950 mobile computing devices via the network 920 (using a wired or wireless connection for example). As mentioned above, this may be undertaken in response to receiving a request from the first 940 or second 950 mobile computing devices.

Based on the received output signals, the first 940 and second 950 mobile computing devices are adapted to display one or more graphical elements in a display area provided by their respective display. For this purpose, the first 940 and second 950 mobile computing devices each comprise a software application for processing, decrypting and/or interpreting received output signals in order to determine how to display graphical elements. Thus, the first 940 and second 950 mobile computing devices each comprise a processing arrangement adapted to determine a subject-specific or tumour-specific complexity score, and to generate a display control signal for modifying at least one of the size, shape, position, orientation, pulsation or colour of a graphical element based on the determined complexity score.

The system can therefore communicate information about the complexity score to users of the first 940 and second 950 mobile computing devices. For example, each of the first 940 and second 950 mobile computing devices may be used to display graphical elements to a medical practitioner, doctor, consultant, technician or caregiver for example.

Implementations of the system of FIG. 9 may vary between: (i) a situation where the data processing system 930 communicates display-ready data, which may for example comprise display data including graphical elements (e.g. in JPEG or other image formats) that are simply displayed to a user of a mobile computing device using conventional image or webpage display (can be web based browser etc.); to (ii) a situation where the data processing system 530 communicates raw data set information that the receiving mobile computing device then processes with the complexity-determining processes to generate a complexity score, and then displays graphical elements based on the determined complexity score and data sources that the complexity score is based on (for example, using local software running on the mobile computing device). Of course, in other implementations, the processing may be shared between the data processing system 930 and a receiving mobile computing device such that data groups generated at data processing system 930 is sent to the mobile computing device for further processing by local dedicated software of the mobile computing device. Embodiments may therefore employ server-side processing, client-side processing, or any combination thereof.

Further, where the data processing system 930 does not ‘push’ information about a subject-specific disease state or progression, but rather communicates information in response to receiving a request, the user of a device making such a request may be required to confirm or authenticate their identity and/or security credentials in order for information to be communicated.

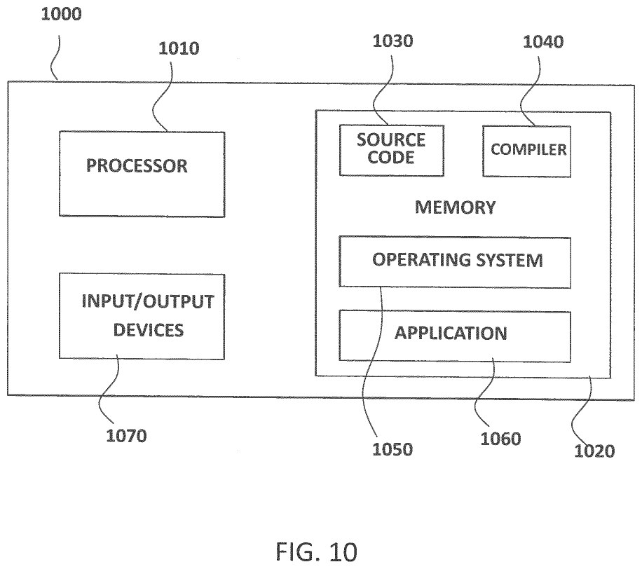

FIG. 10 illustrates an example of a computer 1000 within which one or more parts of an embodiment may be employed. Various operations discussed above may utilize the capabilities of the computer 1000. For example, one or more parts of a system for determining the complexity score of a subject may be incorporated in any element, module, application, and/or component discussed herein.

The computer 1000 includes, but is not limited to, PCs, workstations, laptops, PDAs, palm devices, servers, storages, and the like. Generally, in terms of hardware architecture, the computer 1000 may include one or more processors 1010, memory 1020, and one or more I/O devices 1070 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 1010 is a hardware device for executing software that can be stored in the memory 1020. The processor 1010 can be virtually any custom made or commercially available processor, a central processing unit (CPU), a digital signal processor (DSP), or an auxiliary processor among several processors associated with the computer 1000, and the processor 1010 may be a semiconductor based microprocessor (in the form of a microchip) or a microprocessor.

The memory 1020 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 1020 may incorporate electronic, magnetic, optical, and/or other types of storage media. Note that the memory 1020 can have a distributed architecture, where various components are situated remote from one another, but can be accessed by the processor 1010.

The software in the memory 1020 may include one or more separate programs, each of which comprises an ordered listing of executable instructions for implementing logical functions. The software in the memory 1020 includes a suitable operating system (O/S) 1050, compiler 1040, source code 1030, and one or more applications 1060 in accordance with exemplary embodiments. As illustrated, the application 1060 comprises numerous functional components for implementing the features and operations of the exemplary embodiments. The application 1060 of the computer 1000 may represent various applications, computational units, logic, functional units, processes, operations, virtual entities, and/or modules in accordance with exemplary embodiments, but the application 1060 is not meant to be a limitation.

The operating system 1050 controls the execution of other computer programs, and provides scheduling, input-output control, file and data management, memory management, and communication control and related services. It is contemplated in one embodiment that the application 1060 for implementing exemplary embodiments may be applicable on all commercially available operating systems.

Application 1060 may be a source program, executable program (object code), script, or any other entity comprising a set of instructions to be performed. When a source program, then the program is usually translated via a compiler (such as the compiler 1040), assembler, interpreter, or the like, which may or may not be included within the memory 1020, so as to operate properly in connection with the O/S 1050. Furthermore, the application 1060 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 1070 may include input devices such as, for example but not limited to, a mouse, keyboard, scanner, microphone, camera, etc. Furthermore, the I/O devices 1070 may also include output devices, for example but not limited to a printer, display, etc. Finally, the I/O devices 1070 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 1070 also include components for communicating over various networks, such as the Internet or intranet.

If the computer 1000 is a PC, workstation, intelligent device or the like, the software in the memory 1020 may further include a basic input output system (BIOS) (omitted for simplicity). The BIOS is a set of essential software routines that initialize and test hardware at startup, start the O/S 1050, and support the transfer of data among the hardware devices. The BIOS is stored in some type of read-only-memory, such as ROM, PROM, EPROM, EEPROM or the like, so that the BIOS can be executed when the computer 800 is activated.

When the computer 1000 is in operation, the processor 1010 is configured to execute software stored within the memory 1020, to communicate data to and from the memory 1020, and to generally control operations of the computer 1000 pursuant to the software. The application 1060 and the O/S 1050 are read, in whole or in part, by the processor 1010, perhaps buffered within the processor 1010, and then executed.

When the application 1060 is implemented in software it should be noted that the application 1060 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 1060 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description has been presented for purposes of illustration and description, and is not intended to be exhaustive or limited to the invention in the form disclosed. Many modifications and variations will be apparent to those of ordinary skill in the art. Embodiments have been chosen and described in order to best explain principles of proposed embodiments, practical application(s), and to enable others of ordinary skill in the art to understand various embodiments with various modifications are contemplated.

While the invention has been illustrated and described in detail in the drawings and in the foregoing description, such illustration and description are to be considered illustrative and exemplary, not restrictive. The invention is not limited to the disclosed embodiments.

Other variation to the disclosed embodiments can be understood and effected by those skilled in the art in practicing the claimed invention, from a study of the drawings, the disclosure and the appended claims.

In the claims, the word “comprising” does not exclude other elements or steps, and the indefinite article “a” or “an” does not exclude a plurality.

A single unit, processor or other device may fulfill the functions of several items recited in the claims. The mere fact that certain measures are recited in mutually different dependent claims does not indicate that a combination of these features cannot be used to advantage.

Operations like receiving, determining, displaying and/or mapping performed by one or several units can be performed by any other number of units or devices. These operations and/or the control of the decision support system can implemented as program code by means of a computer program and/or as dedicated hardware, firmware or a combination of the foregoing.

A computer program may be stored/distributed on a suitable medium, such as an optical storage medium or a solid-state medium supplied together with or as part of other hardware, but may also be distributed in other forms, such as via the Internet or other wired or wireless telecommunication system. Any reference signs in the claims should not be construed as limiting the scope.

1. A device for supporting a decision based on a plurality of medical findings, the device comprising:
a receiver for receiving the plurality of medical findings, each medical finding is received from a different data source;
a complexity engine for determining a complexity score indicating a level of certainty for the decision based on the medical findings from the different data sources; and
a display for displaying the medical findings and the complexity score.

2. The device of claim 1, wherein the complexity score is based on determining a level of:
conclusiveness for each of the medical findings; and/or
consensus between the medical findings.

3. The device of claim 1, wherein the complexity score is based on determining a level of conclusiveness for at least one of the medical findings that is most relevant to the decision.

4. The device of claim 1, wherein the display has an icon for indicating the medical findings and complexity score.

5. The device of claim 1, wherein the display has a circular icon for indicating:
each data source;
a number of the data sources used to determine the complexity score by splitting the circular icon into a corresponding number of segments;
the medical findings corresponding to each data source by color-coding the segments; and
the complexity score by displaying the circular icon with a different size.

6. The device of claim 1, wherein the complexity score is based on a weighting applied to the medical findings on which the decision is based.

7. The device of claim 5, wherein greater weighting is given to the medical findings based on a pathology data source as compared to the medical findings based on other data sources.

8. The device of claim 1, wherein each of the medical findings is a diagnosis that a lesion is at least one of malignant, benign or inconclusive.

9. The device of claim 1, wherein at least one of the medical findings is made by a radiologist and a second of the medical findings is made by a pathologist.

10. The device of claim 1, wherein the decision relates to diagnosing whether a disease exists.

11. The device of claim 1, wherein the device further comprises:
a data completeness classification unit for classification of whether data from the data sources is complete in determining the level of certainty of the decision; and
wherein the display is configured to display the classification with the complexity score.

12. The device of claim 1, wherein the decision is a TNM staging decision wherein:
the receiver receives a plurality of medical findings relating to each of a plurality of lesions;
the complexity engine determines a complexity score associated with each lesion based on the medical findings relating to the lesion that is inconclusive; and
wherein the complexity engine is configured to determine an overall complexity score of the TNM staging decision based on a quantity of the complexity scores that are inconclusive.

13. The device of claim 1, further comprising a mapping unit for mapping the icons to corresponding anatomical locations of the lesions and wherein the display is configured to display the icons on an anatomical grid.

14. A method for supporting a decision based on a plurality of medical findings, the method comprising:
receiving a plurality of medical findings, each medical finding being received from a different data source;
determining a complexity score indicating a level of certainty of the decision based on the medical findings from the different data sources; and
displaying the medical findings and the complexity score.

15. A computer program for enabling a processor to perform the method of claim 14.
